(19) United States
(12) Patent Application Publication
(10) Pub. No.: US 2021/0338114 A1
(43) Pub. Date: Nov. 4, 2021

(54) DEVICE FOR QUANTIFYING DEXTERITY

(71) Applicants: INSTITUT NATIONAL DE LA SANTE ET DE LA RECHERCHE MEDICALE (INSERM), Paris (FR); CENTRE NATIONAL DE LA RECHERCHE SCIENTIFIQUE (CNRS), Paris (FR); UNIVERSITE DE PARIS, Paris (FR); SENSIX, Poitiers (FR)

(72) Inventors: Maxime TEREMETZ, Paris (FR); Mathieu BOUCHER, Poitiers (FR); Pavel LINDBREG, Paris (FR); Marc MAIER, Paris (FR)

(21) Appl. No.: 17/281,012

(22) PCT Filed: Oct. 4, 2019
(86) PCT No.: PCT/EP2019/076950

§ 371 Date: Mar. 29, 2021

(30) Foreign Application Priority Data
Oct. 4, 2018 (FR) 1859202

(51) Int. Cl. A61B 5/11; A61B 5/22

(52) U.S. Cl. CPC A61B 5/1125; A61B 5/225; A61B 5/1126

(57) ABSTRACT

The invention concerns a device for quantifying the dexterity of the fingers of a hand, comprising a main body, several systems for measuring the movement and/or the force applied by a finger in a pressure direction, each system for measuring comprising a sensor for deformation, the sensor for deformation comprising a deformable body, characterized in that each system for measuring also comprises a guide bearing, integral with the main body, a shaft having a bearing surface, the bearing surface being in contact with the deformable body, a tube, adapted to slide in translation in the guide bearing and around the shaft in the pressure direction, having a head adapted to fix the finger to the tube, a first spring connecting the bearing to the tube, a second spring connecting the shaft to the tube, the first spring and the second spring being pre-tensioned, so as to pre-tension the deformable body.

### FIELD OF THE INVENTION

The invention relates to a device for quantifying the dexterity of the fingers of a hand, for example by detecting finger movement and/or strength. The device can be used, for example, in the diagnosis or therapy of a stroke, or in neurological, myological or arthrological pathologies affecting the hand.

### STATE OF ART

Impaired manual dexterity is a major public health problem since it leads to an impairment of activities of daily living and a loss of autonomy. Many neurological, orthopedic and rheumatological conditions affect manual dexterity. Examples of neurological conditions include stroke, cerebral palsy, multiple sclerosis, cervical spondylosis, myelopathy, carpal tunnel syndrome and dystonia.

Manual dexterity can be quantified for diagnosis and/or clinical prognosis, or for possible finger rehabilitation.

To this effect, the preceding patent, EP 2 659 835, describes a device for measuring and quantifying the displacement of the fingers and the force exerted by the fingers on the device. The device comprises a plurality of pistons inserted into a main body. Each piston includes a tube and a shaft, with the tube surrounding the shaft of the piston. Each tube is also connected to the shaft by a return means, so that the shaft exerts a force on the load cell upon movement of the tube.

However, the described device has the following drawbacks:
the device does not have a piston for the thumb;
a patient's finger may be unstable once in contact with the head of a piston, resulting in errors in measuring finger movement or force;
measurement of finger force or movement is limited to four fingers of the hand simultaneously;
only the flexion of one finger can be measured by the described device.

### SUMMARY OF THE INVENTION

An aim of the invention is to provide a device that at least partially remedies the aforementioned drawbacks of the prior art.

This aim of the present invention is achieved by a device for quantifying the dexterity of the fingers of a hand, comprising:
a main body, the dimensions of the main body allowing a user to hold the device in the palm of the hand,
a plurality of systems for measuring the movement and/or force applied by a finger along a pressure direction, each system for measuring comprises a sensor for deformation, the sensor for deformation comprising a deformable body,
   characterized in that each system for measuring also comprises :
a guide bearing, integral with the main body,
a shaft having a bearing surface, the bearing surface being in contact with the deformable body,
a tube, adapted to slide in translation in the guide bearing and around the shaft in the pressure direction, having a head adapted to fix the finger to the tube,
a first spring connecting the bearing to the tube,
a second spring connecting the shaft to the tube,
   the first spring and the second spring being pre-tensioned, so as to pretension the deformable body and to maintain the tube in equilibrium in the absence of the finger bearing on the head,
   the first spring and the second spring being each in compression at the equilibrium of the system for measuring,
   this arrangement of the device allowing to realize :
measurements of the finger bending force, by moving the finger towards the deformable body in the pressure direction,
measurements of the extension force of the finger, by moving the finger opposite to the deformable body in the pressure direction.

The invention is advantageously completed by the following features, taken individually or in any technically possible combination thereof:
the shaft has the bearing surface on its lower extremity, said lower extremity being housed in an opening of the deformable body and an upper extremity located in the tube above the bearing,
the bearing surface is a frustoconical cup,
the second spring is guided by the shaft and is fixed to an inner shoulder of the tube,
the first spring is guided by the tube,
the first spring and the second spring have the same spring stiffness,
the device comprises five systems for measuring for the five fingers of the hand (including the thumb), each measuring assembly corresponding to a finger of the hand,
the five systems for measuring are aligned in the main body and the bearing directions of the system for measuring lie in one plane,
the piston head comprises a first magnet, the device comprising a second magnet adapted to be integral with a finger to be magnetized by the first magnet,
the five systems for measuring are arranged consecutively in the main body so as to correspond to the thumb, index finger, middle finger, ring finger and little finger, and wherein, at equilibrium, the heads corresponding to the middle finger and ring finger are arranged at a distance from the main body greater than the distance from the main body to the other heads
the five systems for measuring are arranged consecutively in the main body so as to correspond to the thumb, the index finger, the middle finger, the ring finger and the little finger, and the angle formed by the pressure direction of the system corresponding to the ring finger and the pressure direction of the system corresponding to the little finger is greater than 5° and preferably greater than 8°,
the five systems for measuring are arranged consecutively in the main body so as to correspond to the thumb, the index finger, the middle finger, the ring finger and the little finger, and the angle formed by the pressure direction of the system corresponding to the thumb and by the pressure direction of the system corresponding to the index finger is greater than 100° and preferably greater than 120°,
the edges of the extremities of the shaft, the bearing and the tube are rounded (to improve the guidance of the piston and the regularity of the stroke of the piston tube),
the stiffness and the length of the springs are chosen so as to allow a measurement of force in extension and a measurement of bending over a length of 10 mm, the maximum travel of the tube in the main body being 10 mm,
the deformable body is preloaded with a tensile force of −1 N to allow a measurable force range of 6 N, between −1 N and 5 N, and with a resolution of 0.1 N,
the device allows the measurement of the force of each finger in dynamic flexion (1N range) from equilibrium to maximum travel, the force of each finger in static flexion (isometric, 4N range), when the tube is in fixed support on the bearing surface of the shaft, at maximum clearance ; and/or the force of each finger in dynamic extension (1N range) from equilibrium to minimum travel,
the main body has a flat surface on which are the four systems for measuring for the index, middle, ring and little fingers, and a beveled surface for the system for measuring the thumb.

In other words, the advantages obtained in particular with an example of a mode of realization which is not limiting of all the possibilities of realization can be the following:

### Ergonomics and Hygiene

The device is portable and can be held comfortably in several postures/positions (e.g. sitting with the hand on the thigh, lying in bed with the hand on a table). A rounded support body in the palm and an attachment around the hand (e.g. by scratch) helps to maintain the position of use of the device (without voluntary effort of the user).
A piston for the thumb is added
The position of the systems for measuring (each system for measuring includes a piston) is compatible with the use of the device by the right and left hand. The heads of the pistons for fingers 3 and 4 are higher than the heads of the other pistons (+7 mm) to compensate the difference in length between the fingers.
The fingers are fixed, by magnets, on the pistons to guarantee a continuous contact with the force sensors. The fixation of the fingers can be done by the subject alone (without help) and the fixation is simple to do.
The contact surfaces are easy to clean with a disinfectant.

### Force Measurement

The device allows to measure the force and/or the displacement of the thumb (finger 1).
The device allows a force measurement in flexion (10 mm) and in extension (10 mm). The total displacement can be equal to 20 mm.
The device allows to measure the force in dynamic (range of 2N) and in static (isometric, range of 3N). So in total: 5N in flexion, and 5N in extension.
The force signal is not disturbed by the transition between dynamic and static modes.

### Interface and Games

The device can be used with the tasks described in patent application WO2016184935.
Visual display of targets and user strengths can be in real-time and on a separate, portable medium (tablet, etc.).
Performance feedback per trial can be provided (one sound for success, another sound for failure, at least for tracking, motor memorization, and the multi-finger tapping task).
The device allows to establish the level of each user. A 15-second finger tapping speed test, for example, can be used to determine the tapping rate of the index finger. The result gives 10 levels for the games (1 to 5 taps correspond to level 1, 6 to 10 taps correspond to level 2, and so on, until more than 50 taps correspond to level 10). The levels correspond to the speed of the tasks. For example, in the force tracking task, level 1 corresponds to the same display time as the current one, level 2 corresponds to a display time that is 20% faster than level 1, level 3 corresponds to a display time that is 40% faster than level 1, up to level 10 which corresponds to a display time that is 200% faster than level 1.
Four games are included: force tracking, single finger tapping (tapping in rhythm with visual and auditory feedback), memorization of a motor sequence (five different sequences are proposed), and multifinger tapping (tapping a combination of fingers according to displayed configurations).
The device allows an automatic quantification of the performance measures, transmitted by the display of a score after each task (and calculated according to the error and the release time; the maximum frequency and the timing variation; the percentage of success in the learning phase and the memorization; the percentage of success and the independence of the fingers).

Process WO2016184935 is incorporated by reference to this patent application.

### BRIEF DESCRIPTION OF THE DRAWINGS

Further features and advantages will be apparent from the following description, which is purely illustrative and non-limiting, and should be read in conjunction with the attached figures, among which:

FIG. 1 is a cross-section of a device comprising five systems for measuring,

FIG. 2 illustrates a system for measuring,

FIG. 3, FIG. 4 and FIG. 5 illustrate the same system for measuring in configurations corresponding to equilibrium, finger flexion and finger extension respectively,

FIG. 6 illustrates an enlarged view of a part of a system for measuring comprising a bearing, a tube, and a first spring,

FIG. 7 illustrates a portion of a system for measuring comprising a tube and a shaft, the shaft having a rounded end.

FIG. 8 illustrates an arrangement of five systems for measuring,

FIG. 9 is a cross-sectional view of the tube of a system for measuring, the tube having an internal shoulder,

FIG. 10 illustrates a shaft having a projection for fitting into an opening in the surface of a deformable body.

### GENERAL DESCRIPTION

General Description of the Device 1

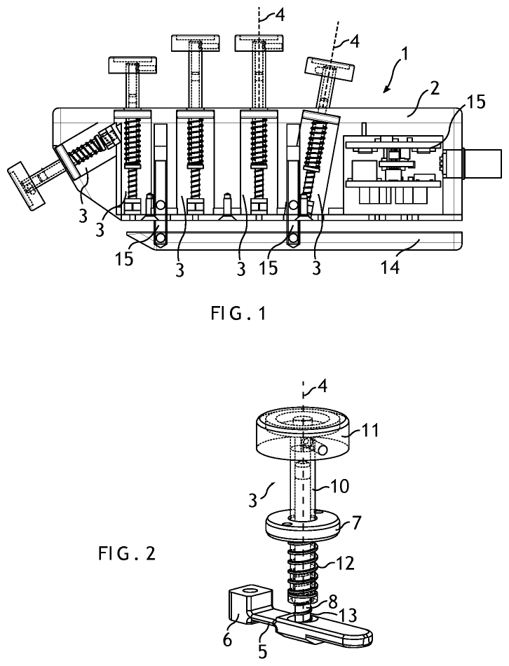

With reference to FIG. 1, the device 1 comprises a main body 2. The dimensions of the main body 2 allow a user to hold the device 1 in the palm of the hand (left hand or right hand). The main body 2 may include a support body 14 that can be held in the palm of the hand. The support body 14 is in the form of a plate. The support body 14 is mechanically connected to the main body 2 by four arms 15, which can be made integral with both the support body 14 and the main body 2. In a different configuration, the support body 14 can be moved by sliding the arms 15 in the main body 2 in order to adapt the device size 1 to the palm of the user's hand. Preferably, the support body 14 may have a rounded shape to be placed against the palm of the user's hand. The main body 2 can also preferably include means for attaching the device 1 to the hand, for example a strap whose size is adaptable by a textile hook and loop attachment.

The device 1 has five systems for measuring 3, partially inserted without the main body 2. Each of the system for measuring 3 may correspond to one of the fingers of the hand. Preferably, four systems for measuring 3 corresponding to the index finger, middle finger, ring finger and little finger are arranged or inserted through the same flat surface of the main body 2. Preferably, the main body 2 also has a beveled surface through which the system for measuring 3 matches to the thumb is inserted.

The device 1 comprises an electronic processing unit 15. The electronic processing unit 15 is adapted to receive and process electrical signals from the various measuring systems 3. The electronic processing unit 15 is also adapted to transmit signals representative of the electrical signals from the various systems for measuring 3 to the outside of the device 1, for example by wire or wireless means.

General Description of a Measuring System 3

With reference to FIG. 2, a system for measuring 3 includes a sensor for deformation 6, for example a strain gauge. The sensor for deformation 6 comprises a deformable body 5. The sensor for deformation 6 is connected to an electronic processing unit 15. The sensor for deformation 6 is adapted to emit an electrical signal representative of a force applied to the deformable body 5.

The system for measuring 3 also includes a guide bearing 7. The guide bearing 7 is integral with the main body 2.

The system for measuring 3 comprises a tube 10. The tube 10 is adapted to slide, for example translationally, in the guide bearing 7 along a pressure direction 4.

The system for measuring 3 comprises a pin 8 (so-called “piston pin”). The tube 10 (the so-called “piston tube”) is at least open at one of its ends, to allow the reception of the shaft 8, so as to form a piston. The tube 10 is adapted to slide around the shaft 8 in the pressure direction 4. The shaft 8 has a bearing surface, not visible in FIG. 2, in contact with the deformable body 5, on which the shaft 8 comes to bear so as to deform the deformable body 5 of the sensor for deformation 6.

The shaft 8 is thus held in a vertical or inclined position in the device 1, solely by the support of the bearing surface 9 on the deformable body 5 by its lower extremity freely housed in an opening of the deformable body 5, and by its holding inside the tube 10.

The system for measuring 3 includes a first spring 12. The first spring 12 is mechanically connected to the guide bearing 7 and the tube 10. Thus, a restoring force connects the guide bearing 7 and the tube 10. By restoring force is meant both an attractive and a repulsive force. The restoring force is considered in its algebraic sense. Preferably, the first spring 12 is guided by the tube 10. In particular, a first end of the spring 12 may be connected to an end of the tube 10 intended to be inserted into the main body 2, and a second extremity of the first spring 12 may be connected to the guide bearing 7.

The system for measuring 3 includes a second spring 13. The second spring 13 is mechanically connected to the shaft 8 and the tube 10. Thus, a restoring force connects the shaft 8 and the tube 10. Preferably, the second spring 13 is guided by the shaft 8. In particular, a first extremity of the second spring 13 may be connected to the extremity of the shaft 8 having the bearing surface 9, and a second end of the second spring 13 may be attached to an inner shoulder of the tube 10. Generally, the first spring 12 and the second spring 13 are arranged to constrain the tube 10 in opposite directions, and preferably in two opposite directions along the bearing direction 4.

The configuration of the first spring 12 and the second spring 13 allows the bearing surface 9 to be moved in a first direction along the pressure direction 4 with respect to an equilibrium position of the bearing surface 9 when the tube 10 is pressed, and to move the bearing surface 9 in a second direction along the bearing direction 4 with respect to the equilibrium position of the bearing surface 9 when the tube 10 is pulled. Thus, this arrangement of the device 1 allows for measurements of the bending force of the finger, by moving the finger towards the deformable body along the pressure direction 4, and measurements of the extension force of the finger, by moving the finger away from the deformable body 5 along the pressure direction 4.

Alternatively, it is possible to reverse the tube 10 and the shaft 8 in the mechanism.

The system for measuring 3 also includes a head 11, integral with the tube 10. The head 11 is adapted to place the finger on the tube 10 and/or to fix the finger on the tube 10. In particular, the head 11 may have a face adapted to contact the finger [feel free to add here the characteristics of this face of the head]. The head 11 preferably comprises a first magnet, the device 1 comprising a second magnet adapted to be integral with a finger and to be magnetized by the first magnet. Thus, the integral assembly formed by the head 11 and the tube 10 can follow the movement or stresses of the finger in flexion and extension. Other means of attachment may be used, such as straps or notches.

Operation of a System for Measuring 3

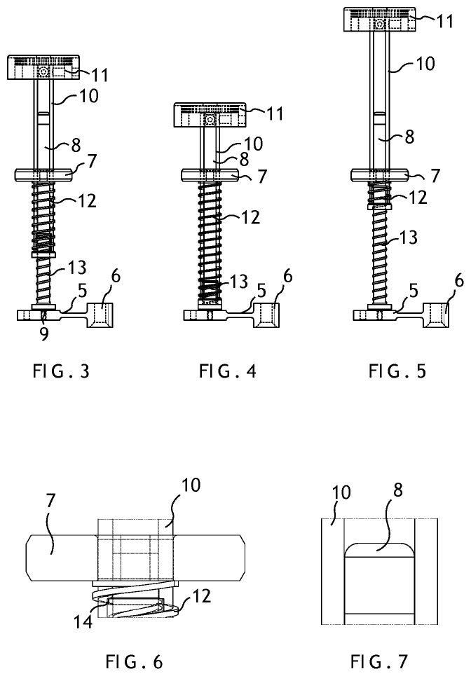

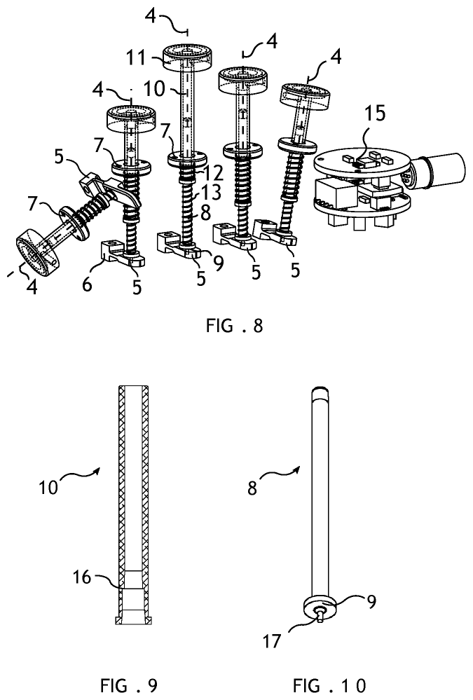

FIG. 3, FIG. 4 and FIG. 5 illustrate respectively configurations of the system for measuring 3 which corresponds to equilibrium, finger flexion and finger extension. By “pre-tensioned” element, we mean that the element undergoes mechanical tension in the equilibrium position of the device 1, without action from a user, potentially leading to a deformation of the element.

With reference to FIG. 3, the first spring 12 and the second spring 13 are preferably preloaded, i.e., the first spring 12 and the second spring 13 are compressed in the equilibrium position of the device 1, when no force external to the device 1 is exerted on the system for measuring system 3, in particular on the tube 10. The preloading of the first spring 12 and the second spring 13 is realized in such a way that the deformable body 5 is also preloaded in the equilibrium position of the device 1. Thus, the deformable body 5 is deformed in the equilibrium position, which allows it to follow the shaft 8 during an extension movement, and thus the sensor for deformation 6 to detect the extension of a finger. Preferably, the deformable body 5 is preloaded with a tensile force equal to −1 N (i.e., a force directed from the axis 8 toward the deformable body 5) to allow a measurable force range of 6 N, between −1 N and 5 N, and with a resolution of 0.1 N.

The preloading of the first spring 12 and the second spring 13 also makes it possible to maintain the tube 10 in equilibrium in the absence of the finger bearing on the head 11. The preloading of the first spring 12 and the second spring 13 also ensures optimal linearity in the relationship between the force exerted by the finger on the system for measuring 3 and the force exerted by the shaft 8 on the sensor for deformation 6.

The shaft 8 has a bearing surface 9. The bearing surface 9 is presented by an extremity of the shaft 8 that is not covered by the tube 10. The bearing surface 9 may be, for example, flat, conical, or formed by a protrusion of the shaft 8. The deformable body 5 preferably has a surface complementary to the bearing surface 9. Thus, it is possible to ensure precise contact between the shaft 8 and the deformable body 5, reducing the mechanical clearances and thus increasing the accuracy of the measurements made by the device 1. The shaft 8 can for example have a bearing surface 9 being a frustoconical cup, preferably with a projection 17 intended to fit into an opening in the surface of the deformable body 5. The shaft 8 has the bearing surface 9 on its lower extremity housed in an opening in the deformable body 5 and an upper end located in the tube above the bearing. In this way, it is possible to avoid play in directions other than the pressure direction 4.

Preferably, the first spring 12 and the second spring 13 have the same stiffness. Thus, the flexion and extension of a finger can be determined with the same sensitivity by the device 1.

Preferably, the first spring 12 and/or the second spring 13 has a stiffness between 0.01 N/mm and 1 N/mm. Thus, the stiffness of the spring(s) is adapted to the force of a finger.

With reference to FIG. 4, a bending finger can exert a tension on the head 11 of the system for measuring 3. This allows the tube 10 to slide around the shaft 8, until the closed end of the tube 10 is in contact with the shaft 8. In this configuration, the first spring 12 is less compressed than in the equilibrium position. The first spring 12 is, in this configuration, still compressed with respect to the equilibrium position of the first spring 12 alone, i.e. considered separately from the device, without external constraint. In this configuration, the second spring 13 is more compressed than in the equilibrium position. The tube 10 may have an internal shoulder 16, arranged at a distance from the extremity of the tube 10 opposite the head 11, corresponding to the maximum compression length of the second spring 13. The second spring 13 can thus be compressed between the internal shoulder 16 and the end of the shaft 8 in contact with the deformable body 5 according to the distance defined by the internal shoulder 16 when the extremity of the tube 10 opposite the head 11 comes to meet the end of the shaft 8. In this configuration, the deformable body 5 is more deformed than in the equilibrium configuration shown in FIG. 3.

With reference to FIG. 5, an extending finger connected to the head 11 can exert a constraint on the head 11. This allows the tube 10 to slide about the shaft 8 away from it. In this configuration, the first spring 12 is more compressed than in the equilibrium position of the system for measuring 3. The second spring 13 is less compressed than in the equilibrium position of the system for measuring 3, and preferably more compressed than in the equilibrium position of the second spring 13 alone, i.e. considered separately from the device, without external constraint. In this configuration, the deformable body is less constrained, and thus less deformed, than in the equilibrium position of the system for measuring 3. Thus, it is possible to measure the extension of a finger.

Preferably, the stiffnesses of the first spring 12, of the second spring 13, as well as the dimensions of the tube 10 and of the shaft 8 are chosen in such a way as to allow a deflection of the tube 10 with respect to the main body 2 of 10 mm with respect to an equilibrium position of the tube 10. This deflection can bring the tube 10 closer or further away from the main body 2 depending on whether the finger is flexed or extended.

With reference to FIG. 6 and FIG. 7, the edges of the various extremities of the parts of the device 1, preferably of the shaft 8, the tube 10 and/or the bearing 7, are rounded. By “rounded”, we mean that the edges have a radius of curvature greater than 50 μm, preferably greater than 100 μm and in particular greater than 150 μm. Thus, the friction between the individual parts, for example between the shaft 8 and the tube 10, is reduced during the movement of the system for measuring 3. FIG. 6 illustrates rounded guide bearing edges 7 and a rounded shoulder 14 in the tube 10. FIG. 7 illustrates a rounded end of the shaft 8.

Arrangement of the Systems for Measuring 3

With reference to FIG. 6, the particular arrangement of the various systems for measuring 3 in the main body 2 solves problems of the prior art.

The pressure directions 4 of the system for measuring 3, in particular of the five systems for measuring 3, lie in one plane. Thus, the systems for measuring 3 are symmetrical with respect to a plane, which allows the device to be adapted to a left hand and a right hand.

The heads 11 corresponding to the middle and ring fingers are arranged at a distance from the main body 2 greater than the distance of the other heads 11 from the main body 2.

The angle formed by the pressure direction 4 of the system for measuring 3 which corresponds to the ring finger and by the pressure direction 4 of the system for measuring 3 which corresponds to the little finger is preferably greater than 5° and in particular greater than 8°. Furthermore, the angle formed by the pressure direction 4 of the system for measuring 3 which corresponds to the thumb and by the pressure direction 4 of the system for measuring 3 which corresponds to the index finger is greater than 100° and preferably greater than 120°.

Thus, the distances between the heads 11 of the system for measuring 3 in equilibrium positions correspond to the distances between the different finger tips at rest in a pronated position.

1. A device for quantifying dexterity of fingers of a hand, comprising:
a main body having dimensions allowing a user to hold the device in a palm of the hand,
a plurality of systems for measuring movement and/or force applied by said fingers along a pressure direction, each system for measuring comprising:
a sensor for deformation, the sensor for deformation comprising a deformable body,
a guide bearing, integral with the main body,
a shaft having a bearing surface, the bearing surface being in contact with the deformable body,
a tube adapted to slide in translation in the guide bearing and around the shaft in the pressure direction, the tube having a head adapted to fix a respective said finger to the tube,
a first spring connecting the bearing to the tube, and
a second spring connecting the shaft to the tube,
the first spring and the second spring being pre-tensioned, so as to pretension the deformable body and to maintain the tube in equilibrium in the absence of pressure of the associated finger on the head,
wherein the first spring and the second spring are each in compression at equilibrium of the system for measuring,
this arrangement of the device allowing to realize:
measurements of finger bending force, by moving the associated finger towards the deformable body in the pressure direction,
measurements of finger extension force, by moving the associated finger opposite to the deformable body in the pressure direction.

2. The device according to claim 1, wherein the shaft has the bearing surface on its lower extremity, said lower extremity being housed in an opening of the deformable body, and an upper extremity of said shaft being located in the tube above the bearing.

3. The device according to claim 1, wherein the bearing surface is a frustoconical cup.

4. The device according to claim 1, wherein the second spring is guided by the shaft and is attached to an inner shoulder of the tube.

5. The device according to claim 1, wherein the first spring is guided by the tube.

6. The device according to claim 1, wherein the first spring and the second spring have the same spring stiffness.

7. The device according to claim 1, comprising five said systems for measuring one for each fingers of the hand.

8. The device according to claim 1, wherein the pressure directions for all the systems for measuring are included in the same plane.

9. (canceled)

10. The device according to claim 8, comprising five said systems for measuring arranged consecutively in the main body so as to correspond to a thumb, index finger, middle finger, ring finger and little finger of the hand, and wherein, at equilibrium, the heads corresponding to the middle finger and the ring finger are arranged at a distance from the main body that is greater than distances from the main body to the other heads.

11. The device according to claims 8, comprising five systems for measuring arranged consecutively in the main body to correspond to a thumb, index finger, middle finger, ring finger and little finger of the hand, and wherein:
an angle formed between the pressure direction of the system for measuring corresponding to the ring finger and the pressure direction of the system for measuring corresponding to the little finger is greater than 5°.

12. The device according to claim 8, comprising five systems for measuring are arranged consecutively in the main body so as to correspond to a thumb, index finger, middle finger, ring finger and little finger of the hand, and wherein:
an angle formed between the pressure direction of the system for measuring corresponding to the thumb and the pressure direction of the system for measuring corresponding to the index finger is greater than 100°.

13. The device according to claim 1, in which the edges of the extremities of the shaft, of the bearing, of the tube are rounded.

14. The device according to claim 1, wherein stiffness and length of the springs are chosen so as to allow a force measurement in extension and a bending measurement over a length of 10 mm, a maximum travel of the tube in the main body being 10 mm.

15. The device according to claim 1, wherein the deformable body is pre-tensioned by a traction force of −1N to allow a measurable force range of 6N, between −1 N and 5 N, and with a resolution of 0.1N.

16. The device according to claim 1, wherein the device allows to measure:
the force of each finger in dynamic bending from equilibrium to maximum clearance;
the force of each finger in static flexion, when the tube is in fixed support on the bearing surface of the shaft for the respective system for measuring, at maximum clearance; and/or
the force of each finger in dynamic extension from equilibrium to minimum clearance.

17. The device according to claim 1, wherein the main body presents:
a flat surface on which are four said systems for measuring for an index finger, a middle finger, a ring finger and a little finger of the hand, and
a beveled surface for a said system for measuring for a thumb of said hand.

18. The device according to claim 11, said angle formed between the pressure direction of the system for measuring corresponding to the ring finger and the pressure direction of the system for measuring corresponding to the little finger being greater than 8°.

19. The device according to claim 12, said angle formed between the pressure direction of the system for measuring corresponding to the thumb and the pressure direction of the system for measuring corresponding to the index finger is greater than 120°.
